(19) United States
(12) Patent Application Publication
(10) Pub. No.: US 2013/0018222 A1
(43) Pub. Date: Jan. 17, 2013

(54) HUMAN GENITALIA STIMULATION DEVICE

(71) Applicants: Vladimir Vitalievich Miroshnichenko, Ljubertsi (RU); Vitaly Evgenievich Pilkin, Moscow (RU)

(72) Inventors: Vladimir Vitalievich Miroshnichenko, Ljubertsi (RU); Vitaly Evgenievich Pilkin, Moscow (RU)

(21) Appl. No.: 13/634,014

(22) PCT Filed: Apr. 4, 2011
(86) PCT No.: PCT/RU11/00219

§ 371 Date: Sep. 11, 2012

(30) Foreign Application Priority Data
Apr. 5, 2010 (RU) 2010112945

(51) Int. Cl. A61F 5/41; A61H 1/00

(52) U.S. Cl. 600/38; 601/46

(57) ABSTRACT

A stimulation device configured to stimulate male's genital organ and female's genital organ comprises the body which has the inner space with the inlet to place in it male's penis, and which (the body) in whole or in part is placed in female's vagina; herewith mechanical stimulation and/or vibratory stimulation and/or electrical stimulation and/or thermal stimulation of male's penis is implemented by technical means placed in the stimulation device and/or by removable sleeve or by removable sleeves; mechanical stimulation and/or vibratory stimulation and/or electrical stimulation and/or thermal stimulation of female's vagina is implemented by technical means placed in the stimulation device and/or by removable sleeve or by removable sleeves.

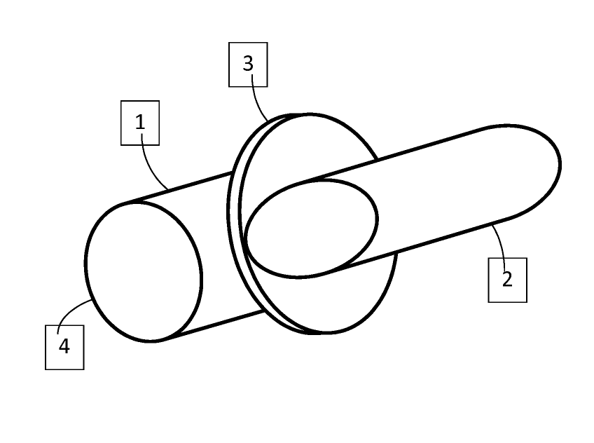

### CROSS-REFERENCE TO RELATED APPLICATIONS

This application is a U.S. national stage application of the PCT application PCT/RU2011/000219 filed on 4 Apr. 2011, whose disclosure is incorporated herein in its entirety by reference, which PCT application claims priority of a Russian Federation application RU2010112945 filed on 5 Apr. 2010.

### FIELD OF THE INVENTION

The invention relates to human genitalia stimulation device for applying in sexual pathology for the purpose of sharing by male and female with functional sexual disorders.

### BACKGROUND OF THE INVENTION

There are known sexual devices used in intimate terms of couples which help to achieve by couples sexual satisfaction at any age, especially with functional sexual disorders (e.g., impotency, frigidity, anorgasmia, etc.) of one or both marital partners. Sexual devices are also used by single people, who for various reasons cannot find partners for sex. More information is available at http://en.wikipedia.org/wiki/Sex_toy. The disadvantage of specified sexual devices is the lack of sense of naturality during implementation of an intimate terms of couple, as foreign objects of sexual orientation are required for sexual satisfaction, that may painfully remind married couple about sexual deficiency of one or both partners, not to bring sexual satisfaction to one of the partners, and thus hurt psyche of married couple as a whole.

The claimers were unable to find in public information sources technical decision that could be considered as a prototype of the invention.

### DESCRIPTION OF THE INVENTION

The task to be solved by the claimed invention is to provide a human genitalia stimulation device for the purpose of sharing by male and female. As a result of using specified invention, the couple in which one or both marital partners suffer functional sexual disorders (impotency, frigidity, anorgasmia, etc.), creates a sense of naturality and harmonious sexual satisfaction during implementation of intimate terms of couple.

Specified technical result is achieved as follows:

A stimulation device designed to stimulate male's genital organ and female's genital organ includes a body in which is placed male's penis, and which (body) in whole or in part is placed in female's vagina.

The body of stimulation device includes two different parts, herewith one part of the body is designed to be placed (in whole or in part) in female's vagina (hereinafter specified part of the stimulation device body is referred to as “female part of the body”), other part of the body is designed to place in it male's penis (hereinafter—“male part of the body”).

The stimulation device body may contain a stopper, movable or not movable relative to the stimulation device body, which does not allow to penetrate the stimulation device body in female's vagina more than prescribed distance.

The stimulation device is configured to implement by technical means, placed in the stimulation device body, and/or by removable sleeve or by removable sleeves:
a) mechanical stimulation of male's penis and/or female's vagina and/or organ or organs relating to external female's genitalia, and/or
b) vibratory stimulation of male's penis and/or female's vagina and/or organ or organs relating to external female genitalia, and/or
c) thermal stimulation of male's penis and/or female's vagina and/or organ or organs relating to external female genitalia, and/or
d) electrical stimulation of male's penis and/or female's vagina and/or organ or organs relating to external female genitalia.

The stimulation device may be configured to implement stimulation of female's genital organ by technical means in terms of:
a) amplitude and/or frequency and/or speed of reciprocating and/or other movements of male's penis inside the male part of the stimulation device body, and/or
b) position or positions of male's penis inside the male part of the stimulation device body, and/or
c) situation of male's penis inside the male part of the stimulation device body, and/or
d) combination or combinations of successive positions of male's penis inside the stimulation device body, and/or
e) size or sizes of male's penis or size or sizes of at least one part of male's penis.

The stimulation device body can be configured with power of disconnection and reverse connection of male and female parts of the stimulation device body.

Stimulation of male's genital organ can be carried by means of the inner surface of the carrying body of stimulation device, including:
a) implementation of multiple pulsed longitudinal increase or decrease of the stimulation device body size (length) and/or single longitudinal increase or decrease of the stimulation device body, and/or
b) implementation of multiple pulsed increase and decrease of external and/or internal diameter of at least one part of the stimulation device body and/or one-time increase of external and/or internal diameter of at least one part of the stimulation device body, and/or
c) programming of specified electrical stimulation by adjustment and/or selection of type and/or amount of current and/or voltage and/or frequency and/or duration of pulses and pauses and/or voltage form and/or electric field configuration, and/or
d) changes of vibrating processes speed at inner and/or outer surface of the lifting body and/or
e) temperature changes in intracavitary sections of stimulation device body surfaces and/or thermoregulation of inner and/or outer surface of the stimulation device body, and/or
f) dynamic dimensional changes of inner and/or outer surfaces of the stimulation device body, and/or
g) coordination of various types of stimulation to other types of stimulation, and/or
h) dynamic changes in the geometric dimensions of at least one intracavitary section of the stimulation device body.

The stimulation device may include at least one power supply, hardware and software for mechanical stimulation and/or vibratory stimulation and/or electrical stimulation and/or thermal stimulation.

In specified stopper can be placed technical means by which implement: mechanical stimulation and/or vibratory stimulation and/or electrical stimulation and/or thermal stimulation of at least one of female external genital organs and/or emission of one or more smell and/or emission of sound or sounds and/or stimulation device control.

The female part of the body:
a) may have diameter that is less than or greater than internal body diameter of the male part of the body, and/or
b) may have length that is less than or greater than body length of the male part of the body, and/or
c) may not have space to place in male's genital organ, and/or
d) can be configured with power to change its length and/or its configuration and/or its thickness, at least in one part and/or relative position of its parts, and/or
e) may have a convex and/or concave shape in at least one of its parts.

### DESCRIPTION OF DRAWINGS

FIGURE attached hereto shows scheme of the stimulation device body, which has male and female parts of the body. 1—the male part of the stimulation device body, 2—the female part of the stimulation device body, 3—stopper, 4—inlet to place in male's penis in the male part of the stimulation device body.

### EMBODIMENT OF THE INVENTION

The invention is feasible, as in the background art technological tools and solutions, including software tools and solutions that allow implementing human genitalia stimulation device according to the invention, are known. Significant features of the invention, expressed in the section “Description of the invention” as an alternative for any choice permitted by this alternative in conjunction with other features characterizing the invention provides receiving the same technical result.

Length and width of internal and external diameter and shape of the male part of the body 1 may be different and/or modifiable.

Length and width of diameter and shape of the female part of the body 2 may be different and/or modifiable.

The male part of the body 1, the female part of the body 2, stopper 3 can be performed of different materials.

At least one part of surface of male part of the body 1 and/or female part of the body 2 and/or stopper 3 may have convex-concave structure and/or heterogeneous structure and/or form.

Inlet 4 may be different sizes, including changing diameter and shape.

Stopper 3 can be rigidly connected to the body of stimulation device (i.e. stopper cannot be moved relative to the stimulation device body) or loosely connected (i.e. stopper can be moved relative to the stimulation device body).

Also feasibility, when stopper 3 may be removable relative to the stimulation device body, and as a result stimulation device body may not have the stopper 3 at all, is considering.

Size and/or shape of stopper 3 may be different and/or modifiable.

Stopper 3 either can move relative to the stimulation device body, or cannot move relative to the stimulation device body.

When moving the stopper 3 relative to the stimulation device body, stopper 3 can be fixed relative to the stimulation device body.

In the stopper 3 may be placed technical means to implement at least one of specified stimulation, with possible stimulation of at least one part of genitalia of female or male.

Also an option when stopper 3 have light fixture and/or means by which the smell and/or means by which the sounds are emitted.

The male part of the body 1 is connected to the female part of the body 2, or between the male part of the body 1 and the female part of the body 2 is part of the stimulation device body, which does not relate to the female part of the body 2 and does not relate to the male part of the body 1.

### APPLICATION OF THE INVENTION

The invention can be applied by couples in which one or both marital partners suffer functional sexual disorders.

1. A stimulation device configured to stimulate male's genital organ and female's genital organ comprises the body which has the inner space with the inlet to place in it male's penis, and which (the body) in whole or in part is placed in female's vagina; herewith mechanical stimulation and/or vibratory stimulation and/or electrical stimulation and/or thermal stimulation of male's penis is implemented by technical means placed in the stimulation device and/or by removable sleeve or by removable sleeves; mechanical stimulation and/or vibratory stimulation and/or electrical stimulation and/or thermal stimulation of female's vagina is implemented by technical means placed in the stimulation device and/or by removable sleeve or by removable sleeves.

2. The stimulation device according to claim 1, wherein the stimulation device body contains a stopper, movable or not movable relative to the stimulation device body, which does not allow to penetrate the stimulation device body in female's vagina more than prescribed distance.

3. The stimulation device according to claim 1, wherein stimulation of male's genital organ is carried by means of the inner surface of the carrying body of stimulation device, including:
a) implementation of multiple pulsed longitudinal increase or decrease of the stimulation device body size (length) and/or single longitudinal increase or decrease of the stimulation device body, and/or
b) implementation of multiple pulsed increase and decrease of external and/or internal diameter of at least one part of the stimulation device body and/or one-time increase of external and/or internal diameter of at least one part of the stimulation device body, and/or
c) programming of specified electrical stimulation by adjustment and/or selection of type and/or amount of current and/or voltage and/or frequency and/or duration of pulses and pauses and/or voltage form and/or electric field configuration, and/or
d) changes of vibrating processes speed at inner and/or outer surface of the lifting body and/or
e) temperature changes in intracavitary sections of stimulation device body surfaces and/or thermoregulation of inner and/or outer surface of the stimulation device body, and/or
f) dynamic dimensional changes of inner and/or outer surfaces of the stimulation device body, and/or
g) coordination of various types of stimulation to other types of stimulation, and/or
h) dynamic changes in the geometric dimensions of at least one intracavitary section of the stimulation device body.

4. The stimulation device according to claim 1, wherein the female part of the stimulation device body:
a) has diameter that is less than or greater than internal body diameter of the male part of the body, and/or
b) has length that is less than or greater than body length of the male part of the body, and/or
c) does not have space to place in male's genital organ, and/or
d) is configured with power to change its length and/or its configuration and/or its thickness, at least in one part and/or relative position of its parts, and/or
e) has a convex and/or concave shape in at least one of its parts.

5. The stimulation device according to claim 1, wherein the stimulation device is configured to implement stimulation of female's genital organ by technical means in terms of:
a) amplitude and/or frequency and/or speed of reciprocating and/or other movements of male's penis inside the male part of the stimulation device body, and/or
b) position or positions of male's penis inside the male part of the stimulation device body, and/or
c) situation of male's penis inside the male part of the stimulation device body, and/or
d) combination or combinations of successive positions of male's penis inside the stimulation device body, and/or
e) size or sizes of male's penis or size or sizes of at least one part of male's penis.

6. The stimulation device according to claim 2, wherein in specified stopper is placed technical means by which are implemented mechanical stimulation and/or vibratory stimulation and/or electrical stimulation and/or thermal stimulation of at least one of female external genital organs and/or emission of one or more smell and/or emission of sound or sounds and/or stimulation device control.

7. The stimulation device according to claim 1, wherein the stimulation device includes at least one power supply, hardware and software for mechanical stimulation and/or vibratory stimulation and/or electrical stimulation and/or thermal stimulation.

8. The stimulation device according to claim 1, wherein the stimulation device body is configured with power of disconnection and reverse connection of male and female parts of the stimulation device body.
